(19) United States
(12) Patent Application Publication
(10) Pub. No.: US 2015/0147385 A1
(43) Pub. Date: May 28, 2015

(54) INTRAVENOUS SYNTHETIC CURCUMIN (S-CURCUMIN) FOR THE TREATMENT OF PROLIFERATIVE DISORDERS

(71) Applicant: SignPath Pharma, Inc., Quakertown, PA (US)

(72) Inventor: Lawrence Helson, Quakertown, PA (US)

(21) Appl. No.: 14/550,672

(22) Filed: Nov. 21, 2014

Related U.S. Application Data
(60) Provisional application No. 61/907,825, filed on Nov. 22, 2013.

(51) Int. Cl. A61K 9/127; A61K 47/48; A61K 45/06; A61K 31/12

(52) U.S. Cl. CPC A61K 9/127; A61K 31/12; A61K 47/48223; A61K 45/06; A61K 47/48215; A61K 47/48207; A61K 47/482
USPC 424/450; 514/679; 424/130.1; 514/567; 514/565; 514/250; 514/17.7

(57) ABSTRACT

Compositions and methods for the treatment of proliferative disorders including proliferative such as breast, uterine cervical, ophthalmic, and pancreatic cancer by the administration of intravenous synthetic curcumin (S-curcumin) are disclosed herein.

### CROSS-REFERENCE TO RELATED APPLICATIONS

This application claims priority to U.S. Provisional Application Ser. No. 61/907,825 filed Nov. 22, 2013, the entire contents of which are incorporated herein by reference.

### STATEMENT OF FEDERALLY FUNDED RESEARCH

None.

### INCORPORATION-BY-REFERENCE OF MATERIALS FILED ON COMPACT DISC

None.

### TECHNICAL FIELD OF THE INVENTION

The present invention relates in general to the field of treatment of proliferative disorders, and more particularly to administering topically, or systemically a therapeutic amount of synthetic curcumin (S-curcumin) to a subject afflicted with proliferative disorders such as breast, uterine cervical, ophthalmic, and pancreatic cancer.

### BACKGROUND OF THE INVENTION

Without limiting the scope of the invention, its background is described in connection with the use of pharmaceutical compositions comprising curcumin, synthetic curcumin and analogues and derivatives thereof.

U.S. Pat. No. 7,220,438 (Quintanilla et al. 2007) discloses a topical pharmaceutical composition comprising an water soluble Curcuma extract, and suitable excipients for said topical administration; the process for obtaining said pharmaceutical compositions; the use of different Curcuma extracts as photosensitizing agents for the treatment of proliferative diseases; and the use of Curcuma extract or curcuminoids in combination with a radiation for the treatment of proliferative diseases on eukaryote cells.

U.S. Patent Application Publication No. 20090047371 (Turini and Coccoloni, 2009) discloses a pharmaceutical composition containing curcumin and resveratrol and its application in the medical field. In particular, the composition according to the invention can be advantageously employed for preventing aging and vascular diseases, for the treatment and the prophylaxis of cancers as prostate carcinoma, of skin diseases as psoriasis, and of the piliferous system as hair loss.

U.S. Patent Application Publication No. 20100286585 (Dimauro et al. 2010) relates to a method for reducing or preventing a human brain disorder relating to the presence of a pathogenic substance in cerebrospinal fluid by selecting a human for treatment as a patient and placing a proximal end of a first catheter, having at least a first lumen, in a first sub-dural location within the brain of the patient to establish open communication between the first lumen and cerebrospinal fluid of the patient. For an extended period of time, a curcumin agent selected from at least one of curcumin, a curcumin hybrid and a curcumin analog is delivered to the cerebrospinal fluid to interact with the pathogenic substance to attenuate its effect on the brain.

### SUMMARY OF THE INVENTION

The present invention relates to pharmaceutical compositions and treatment methods comprising synthetic curcumin (S-curcumin). The invention further describes the use of the pharmaceutical composition for the treatment of proliferative disorders. In one embodiment, the present invention includes a composition for ameliorating symptoms or treating one or more proliferative disorders in a subject comprising: one or more spherical liposomes comprising a lipid or a phospholipid wall, wherein the liposome encloses a synthetic curcumin (S-curcumin) or derivatives and modifications thereof dissolved or dispersed in an aqueous or a non-aqueous solvent with one or more optional related co-factors, proteins, antibodies, pain medications, and other pharmaceutically active agents dissolved, dispersed, or suspended in the solvent; a suitable aqueous or non-aqueous dispersion medium, wherein the one or more spherical liposomes are dispersed in the dispersion medium; and one or more optional excipients, diluents, extended or controlled release agents, lubricants, preservatives or any combinations thereof. In one aspect, the one or more proliferative disorders are selected from the group consisting of breast, uterine, cervical, ophthalmic, pancreatic, or any combinations thereof. In one aspect, the lipid or the phospholipid is selected from the group consisting of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl stearate, isopropyl myristate, amphoteric acrylic polymers, fatty acid, fatty acid amides, cholesterol, cholesterol ester, diacylglycerol, and diacylglycerolsuccinate. In another aspect, the composition is adapted for enteral, parenteral, intravenous, sub-cutaneous, intra-muscular, or intra-peritoneal injection in the subject. In another aspect, the one or more liposomes have a size of about 100 nm. In another aspect, the present invention includes a composition for ameliorating symptoms or treating one or more proliferative disorders in a subject comprising: a biodegradable polymer conjugate dissolved or dispersed in a suitable aqueous or non-aqueous solvent, wherein the conjugate comprises a synthetic curcumin (S-curcumin) or derivatives and modifications thereof conjugated to one or more polymers selected from the group consisting of polyesters, polylactides, polyglycolides, polycaprolactones, polyanhydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), copolymers, terpolymers, and combinations or mixtures thereof; and one or more excipients, diluents, extended or controlled release agents, lubricants, preservatives or any combinations thereof. In another aspect, the one or more proliferative disorders are selected from the group consisting of breast, uterine, cervical, ophthalmic, pancreatic, or any combinations thereof. In another aspect, the composition adapted for enteral, parenteral, intravenous, sub-cutaneous, intra-muscular, or intra-peritoneal injection in the subject. In another aspect, the composition is used for the treatment of one or more neurological or neurodegenerative conditions selected from the group consisting of Parkinson's disease (PD), Alzheimer's disease, stress disorders, senile dementia, vascular dementias, Pick's disease, Creutzfeldt-Jacobs disease, and aging. In another aspect, the synthetic curcumin is 85, 86, 87, 88, 89, 90, 91, 92, 93, 94, 95 or 96% pure diferuloylmethane.

In another embodiment, the present invention includes a method of ameliorating symptoms or treating one or more proliferative disorders in a subject comprising the steps of: identifying the subject in need of amelioration of symptoms or treatment of the one or more proliferative disorders; and administering intravenously one or more pharmaceutical compositions comprising a therapeutically effective amount of a synthetic curcumin (S-curcumin) or derivatives and modifications thereof dissolved or dispersed in a suitable aqueous or non-aqueous medium, wherein the curcumin is enclosed in one or more spherical liposomes or is conjugated to one or more biodegradable polymers. In one aspect, the one or more proliferative disorders are selected from at least one of breast, uterine, cervical, ophthalmic, pancreatic, or any combinations thereof. In another aspect, the liposomes comprise a lipid or a phospholipid wall. In another aspect, the lipid or the phospholipid is selected from the group consisting of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl stearate, isopropyl myristate, amphoteric acrylic polymers, fatty acid, fatty acid amides, cholesterol, cholesterol ester, diacylglycerol, and diacylglycerolsuccinate. In another aspect, the therapeutically effective amount comprises 50 nM/kg, 10 to 100 nM/kg, 25 to 75 nM/kg, 10, 20, 30, 40, 50, 60, 70, 80, 90, or 100 nM/kg of body weight of the subject. In another aspect, the synthetic curcumin is 85, 86, 87, 88, 89, 90, 91, 92, 93, 94, 95 or 96% pure diferuloylmethane.

In another embodiment, the present invention includes a composition for ameliorating symptoms or treating breast cancer in a human subject comprising synthetic curcumin (S-curcumin) or derivatives and modifications thereof dissolved or dispersed in a suitable aqueous or non-aqueous medium, wherein the S-curcumin is enclosed in one or more spherical liposomes or is conjugated to one or more biodegradable polymers. In one aspect, the liposome comprises a lipid or phospholipid, wherein lipid or the phospholipid is selected from the group consisting of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl stearate, isopropyl myristate, amphoteric acrylic polymers, fatty acid, fatty acid amides, cholesterol, cholesterol ester, diacylglycerol, and diacylglycerolsuccinate. In another aspect, the biodegradable polymer is selected from the group consisting of polyesters, polylactides, polyglycolides, polycaprolactones, polyanhydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), copolymers, terpolymers, and combinations or mixtures thereof. In another aspect, the composition adapted for intravenous, sub-cutaneous, intra-muscular, or intra-peritoneal injection in the subject. In another aspect, the present invention includes a composition for ameliorating symptoms or treating at least one of proliferative disorders, neurological disorders, or neurodegenerative conditions, in a subject comprising: curcumin, synthetic curcumin (S-curcumin), curcumin analogues, curcumin derivatives or combinations thereof dissolved or dispersed in a suitable aqueous or non-aqueous medium, wherein the curcumin or the S-curcumin is enclosed in one or more spherical liposomes or is conjugated to one or more biodegradable polymer, wherein the liposome comprises a lipid or phospholipid, wherein lipid or the phospholipid is selected from the group consisting of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl stearate, isopropyl myristate, amphoteric acrylic polymers, fatty acid, fatty acid amides, cholesterol, cholesterol ester, diacylglycerol, and diacylglycerolsuccinate and wherein the biodegradable polymer is selected from the group consisting of polyesters, polylactides, polyglycolides, polycaprolactones, polyanhydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), copolymers, terpolymers, and combinations or mixtures thereof. In another aspect, the one or more proliferative disorders are selected from the group consisting of breast, uterine, cervical, ophthalmic, pancreatic, or any combinations thereof. In another aspect, the neurological disorder or the neurodegenerative condition is selected from the group consisting of Parkinson's disease (PD), Alzheimer's disease, stress disorders, post traumatic stress disorder (PTSD), senile dementia, vascular dementias, Pick's disease, Creutzfeldt-Jacobs disease, and aging. In another aspect, the composition adapted for enteral, parenteral, intravenous, sub-cutaneous, intra-muscular, or intra-peritoneal injection in the subject. In another aspect, the composition is administered along with related co-factors, proteins, antibodies, pain medications, and other pharmaceutically active agents selected from at least one of L-dopa, Carbidopa, benserazide, Tolcapone, dopamine agonists bromocriptine, pergolide, pramipexole, ropinirole, piribedil, cabergoline, apomorphine, lisuride, MAO inhibitors, serotonin reuptake inhibitors, sertraline, paroxetine, selegiline, or rasagiline. In another aspect, the synthetic curcumin is 85, 86, 87, 88, 89, 90, 91, 92, 93, 94, 95 or 96% pure diferuloylmethane.

In another embodiment, the present invention includes a method of ameliorating symptoms or treating one or more proliferative disorders, neurological disorders, neurodegenerative conditions, or any combinations thereof in a subject comprising the steps of: identifying the subject in need of amelioration of symptoms or treatment of the one or more proliferative disorders, neurological disorders, neurodegenerative conditions, or any combinations thereof; and administering intravenously one or more pharmaceutical compositions comprising a therapeutically effective amount of curcumin, synthetic curcumin (S-curcumin), curcumin analogues, curcumin derivatives or combinations thereof dissolved or dispersed in a suitable aqueous or non-aqueous medium, wherein the curcumin or the S-curcumin is enclosed in one or more spherical liposomes or is conjugated to one or more biodegradable polymers. In one aspect, the neurological disorder or the neurodegenerative condition is selected from the group consisting of Parkinson's disease (PD), Alzheimer's disease, stress disorders, post traumatic stress disorder (PTSD), senile dementia, vascular dementias, Pick's disease, Creutzfeldt-Jacobs disease, and aging. In another aspect, the synthetic curcumin is 85, 86, 87, 88, 89, 90, 91, 92, 93, 94, 95 or 96% pure diferuloylmethane.

### BRIEF DESCRIPTION OF THE DRAWINGS

For a more complete understanding of the features and advantages of the present invention, reference is now made to the detailed description of the invention along with the accompanying figures and in which:

FIG. 1 shows wound healing (inhibition of growth) of human breast cancer cells between S-curcumin-PLGA and C3-curcumin PLGA at a 12 hour exposure time in the breast cancer cell line MCF10AT; and

FIG. 2 shows wound healing (inhibition of growth) of human breast cancer cells between

S-curcumin-PLGA and C3-curcumin PLGA at a 12 hour exposure time in the breast cancer cell line MCF10ACA1H cell line.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term “Curcumin (diferuloyl methane; 1,7-bis(4-hydroxy-3-methoxyphenyl)-1,6-heptadiene-3,5-dione)” is a naturally occurring compound which is the main coloring principle found in the rhizomes of the plant Curcuma longa (U.S. Pat. No. 5,679,864 (Krackov et al.)). In one aspect, the synthetic curcumin is 85, 86, 87, 88, 89, 90, 91, 92, 93, 94, 95 or 96% pure diferuloylmethane.

The term “liposome” refers to a capsule wherein the wall or membrane thereof is formed of lipids, especially phospholipid, with the optional addition therewith of a sterol, especially cholesterol.

As used herein, the term “in vivo” refers to being inside the body. The term “in vitro” as used in the present application is to be understood as indicating an operation carried out in a non-living system.

As used herein, the term “treatment” refers to the treatment of the conditions mentioned herein, particularly in a patient who demonstrates symptoms of the disease or disorder.

As used herein, the term “treatment” or “treating” refers to any administration of a compound of the present invention and includes (i) inhibiting the disease in an animal that is experiencing or displaying the pathology or symptomatology of the diseased (i.e., arresting further development of the pathology and/or symptomatology) or (ii) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

The terms “effective amount” or “therapeutically effective amount” described herein means the amount of the subject compound that will elicit the biological or medical response of a tissue, system, animal or human that is being sought by the researcher, veterinarian, medical doctor or other clinician. In one example, the therapeutically effective amount comprises 50 nM/kg, 10 to 100 nM/kg, 25 to 75 nM/kg, 10, 20, 30, 40, 50, 60, 70, 80, 90, or 100 nM/kg of body weight of the subject.

The terms “administration of” or “administering a” compound as used herein should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as intravenous (IV), intramuscular (IM), or intraperitoneal (IP), and the like; enteral or parenteral, transdermal dosage forms, including creams, jellies, powders, or patches; buccal dosage forms; inhalation powders, sprays, suspensions, and the like; and rectal suppositories.

As used herein the term “intravenous administration” includes injection and other modes of intravenous administration.

The term “pharmaceutically acceptable” as used herein to describe a carrier, diluent or excipient must be compatible with the other ingredients of the formulation and not deleterious to the recipient thereof

The present invention discloses compositions and methods for the treatment of proliferative disorders using synthetic curcumin (S-curcumin).

Curcumin is the active principle of the turmeric plant which has been synthesized to near purity (99.2%).¹ It is formulated with liposomes², polymers³, or PLGM to render it capable of being administered intravenously as a bolus or as a continuous infusion over 1-72 hours in combination with other active agents (for e.g. a calcium channel blocker). Curcumin has antioxidant and anti-inflammatory activity, and can block autonomous intracellular signaling pathways abnormally responsive to extracellular growth factors, uncontrolled proliferation of cells and fibrosis-associated and tissue degenerative conditions. Specifically, Curcumin reacts negatively with components of key signaling pathways commanding proliferation, metabolism, survival and death.

Oral and topical administration of the extract of the turmeric plant has been used in traditional medicine for over two thousand years. While oral administration is devoid of systemic toxicity it is also devoid of systemic therapeutic activity.⁵ This is due to blood insolubility, and intestinal wall and hepatic inactivation, i.e. it has negligible bioavailability for systemic diseases by the oral route. To overcome these limitations, parenteral intravenous curcumin formulations with liposomes², polymers³ (n-isopropylacrylamide, N-vinylpyrrolidione and acrylic acid) and polylactic glycolic acid copolymer were entered into in pre-clinical drug development.⁴

Curcumin as an extract of turmeric root is available to researchers as a mixture of three curcuminoids and to the public as a food supplement or spice according to the FDA. The extract is 79.2% curcumin (diferuloylmethane), 18.27% demethoxycurcumin, and 2.53% bisdemethoxycurcumin.

Synthesized curcumin is GMP grade 99.2% pure diferuloylmethane produced for non-human experimental study and future Phase I clinical trials. There are obvious differences between the C3 three component extract and the single component synthesized S-curcumin that extend to discernable analytic, physicochemical, and biological characteristics. In certain aspects, the diferuloylmethane is 85, 86, 87, 88, 89, 90, 91, 92, 93, 94, 95 or 96% pure diferuloylmethane.

The present invention relates to synthetic curcumin (S-curcumin) and compares the properties and the activity of S-curcumin with liposomal curcumin, Nanocurc®, and PLGA-curcumin (hereinafter C3-complex).

Liposomal curcumin: The initial studies of liposomal curcumin were done using material bought as the complex.⁶⁻⁷ Studies with S-curcumin are Mach CM, et al (2009)⁸ and Mach CM et al (2010)⁹.

Nanocurc®: The initial study of Nanocurc® was done using product bought as the complex Savita Bisht et al (2007)¹⁰ used a non-sabinsa source. Since then studies with S-curcumin are used in the remainder of Nanocurc® publications.¹¹⁻¹³

PLGA-curcumin: The initial studies of PLGA-curcumin were done using product manufactured as the C3-complex.¹⁴⁻¹⁸ Studies included PLGA -curcumin C3 complex and PLGA-S-curcumin pharmacokinetic studies in rat brains.

Comparison of PLGA C3-complex-curcumin vs PLGA S-curcumin indicated the following differences. The solubilities of 99.2% S-Curcumin in all four solvents. Ethanol, Ethyl acetate, Acetone, and Acetonitrile, differed significantly from the C3 complex containing 76% curcumin. When normalized to equal concentrations, the pure material has greater solubility. This confers improved manufacturing capability, and attributes to different pharmacokinetics and pharmacodynamics in in vivo settings (Table 1).

TABLE 1
Solubilities of S-curcumin and C3-complex
curcumin in different organic solvents.

| | | | | | |
|---|---|---|---|---|---|
| | | | | Physical | |
| | Wt. of | Vol. of | | appear- | 1 g of solvent |
| | Curcumin | solvent | Conc. | ance of | solubilize in |
| Solvent | (mg) | (μL) | (mg/mL) | solubility | solvent (mL) |

Batch -C100609, Curcumin = 99.2%

| | | | | | |
|---|---|---|---|---|---|
| Ethyl | 20.8 | 2000 | 10.4 | Partial | 96.2 |
| acetate | | | | | |
| Ethanol | 17.5 | 4600 | 3.804 | Partial | 263 |
| Acetone | 46.5 | 1100 | 42.3 | Fully | 23.6 |
| Acetoni- | 17.4 | 3200 | 5.44 | Partial | 184 |
| trile | | | | | |

Batch -C3 complex, Curcumin = 76%

| | | | | | |
|---|---|---|---|---|---|
| Ethyl | 21.5 | 1800 | 11.9 | Fully | 83.7 |
| acetate | | | | | |
| Ethanol | 28.4 | 6500 | 4.37 | Fully | 229 |
| Acetone | 39.8 | 700 | 56.9 | Fully | 17.6 |
| Acetoni- | 18.2 | 2900 | 6.28 | Fully | 159 |
| trile | | | | | |

*Weight the sample and transfer into a scintillation vial. Add small volume of solvent and shake well repeat for each addition till the solubility.

A comparison of differences based upon Certificates of Analyses of between S-Curcumin and C3-Complex curcumin is presented in Table 2.

TABLE 2
A comparison of differences based upon Certificates of
Analyses of between S-Curcumin and C3-Complex curcumin.

| | |
|---|---|
| C3-curcumin | S-curcumin |

| | | |
|---|---|---|
| Residue on Ignition | 0.03% | 0.4% |
| Loss on drying | 0.98% | 0.24% |
| Melting range | 172-175 C. | 179.8-181.9 C. |

| | | | | |
|---|---|---|---|---|
| Tapped bulk density | 0.61 | g/ml | 0.26 | g/ml |
| Loose bulk density | 0.37 | g/ml | 0.18 | g/ml |
| Sieve test | | | | |

| | | |
|---|---|---|
| −40 mesh | 100% | 64.71% |
| −80 mesh | 98.27% | 64.0% |
| HPLC content curcumin | 79.2% | 99.2% |
| Total curcuminoids | 96.21% | 99.2% |
| Bisdemethoxycurcumin | 2.53% | 0 |
| Demethoxycurcumin | 18.27 | 0 |

| | | | | |
|---|---|---|---|---|
| Heavy Metals | | | | |
| Lead | 0.91 | ppm(ug/g) | <0.2 | ppm |
| Arsenic | 0.54 | ppm(ug/g) | <0.2 | ppm |

| | | | |
|---|---|---|---|
| Cadmium | <0.2 | ppm(ug/g) | — |
| Mercury | <0.02 | ppm(ug/g) | — |

| | | |
|---|---|---|
| Residual solvents | complies | complies |

| | | | | |
|---|---|---|---|---|
| Micro -total Plate count | <100 | cfug | 10 | cfu/g |
| Yeast and mold count | <10 | cfu/g | 15 | cfu/g |
| Esherichia coli | neg/10 | g | neg/10 | g |
| Salmonella | neg/10 | g | neg/10 | g |
| Staphaureus | neg/10 | g | neg/10 | g |
| Pseudomonas aeruginosa | neg/10 | g | neg/10 | g |

These data indicate that both the C3 complex of curcuminoids and S-curcumin comply with reference standards, and are within specifications for USP and ICOPOES. There are similarities in heavy metals, residual solvents, and bacterial contamination, however, the remainder of the analytical constituents exhibit discernable differences. The extent to which these differences contribute to differing biological effects is unknown.

Distribution in site difference and measurements are shown in Table 3. Intravenous C3-curcumin PLGA formulations were characterized by significant elevations in background noise during HPLC assays of blood and brain tissues rendering analysis impossible. By contrast, 5-curcumin PLGA was unaccompanied by background noise.

TABLE 3
Mean Brain tissue and Plasma Curcumin Levels in Rats
Following Intravenous PLGA-curcumin: 20 mg/kg.

| | Post-injection | | Brain/Plasma ratio | |
|---|---|---|---|---|
| | 2 hour | 4 hour | 2 hour | 4 hour |
| Striatum (ng/g) | 23.3 ± 13.4 | 0 | 2.8 | . . . |
| Hippocampus (ng/g) | 5.2 ± 8.2 | 5.1 ± 5.2 | 0.63 | 1.1 |
| Brain Stem (ng/gm) | 5.3 ± 5.4 | 0 | 0.65 | . . . |
| Plasma (ng/ml) | 8.2 ± 4.5 | 4.5 ± 4.6 | | |

Values are expressed as mean + SEM. n = 3/group.
For curcumin-PLGA, the purity of free curcumin is 99.2% and the relative ratio of free curcumin to the PLGA copolymer is 1:9, hence the intravenous dose of free curcumin in the CurcuminPLGA formulation is 2 mg/kg.

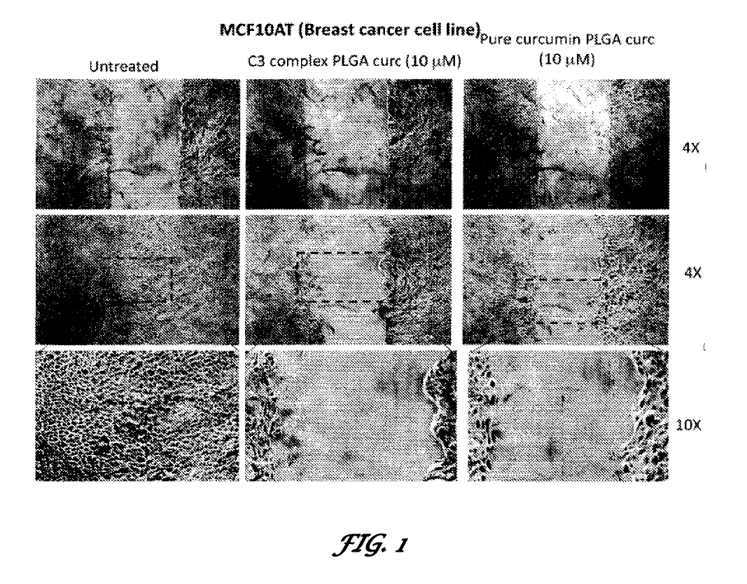

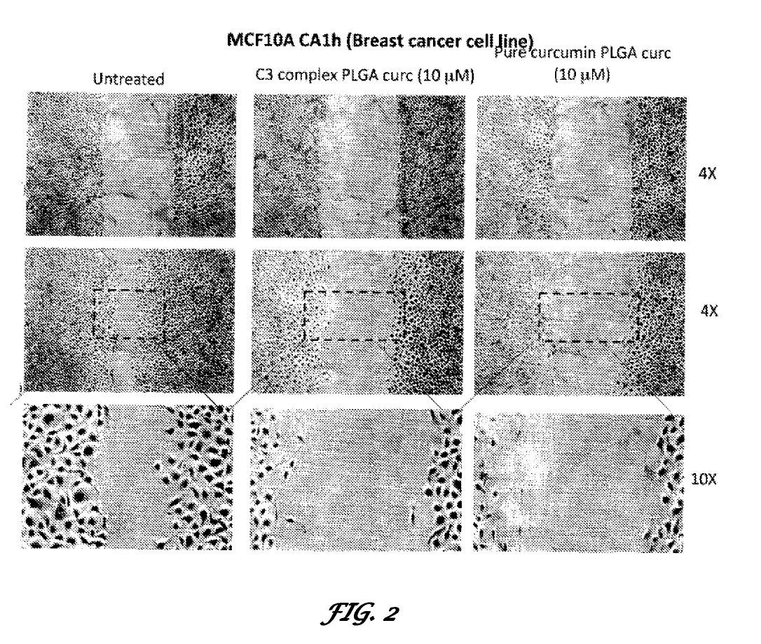

Scratch assay: The lack of a significant difference in interference with wound healing (inhibition of growth) of human breast cancer cells between S-curcumin-PLGA and C3-curcumin PLGA at a 12 hour exposure time in the breast cancer cell line MCF10AT appears to be due to the sensitivity to curcumin of this cell line (Table 4 and FIG. 1). In the MCF10ACA1H cell line (Table 5 and FIG. 2) the S-curcumin is 30% more effective than C3-curcumin in interfering with wound healing.

TABLE 4
Differences in wound healing between S-curcumin-
PLGA and C3-curcumin PLGA at a 12 hour exposure
time in the breast cancer cell line MCF10AT.

| | | C3 complex | Pure curcumin |
|---|---|---|---|
| | | PLGA Curc | PLGA Curc |
| Scratching | Untreated | (curcumin - 76.07%) | (curcumin - 99.2%) |
| Area after | 6433842 | 6467329 | 6760641 |
| scratching | | | |
| t = 0 | | | |
| Area after | 1542075 | 5000390 | 5692562 |
| t = 12 hrs | | | |
| Percentage | 76 | 22 | 15.7 |
| wound | | | |
| healing | | | |

TABLE 5
Differences in wound healing between S-curcumin-
PLGA and C3-curcumin PLGA at a 12 hour exposure
time in the breast cancer cell line MCF10ACA1H.

| | | C3 complex | Pure curcumin |
|---|---|---|---|
| | | PLGA Curc | PLGA Curc |
| Scratching | Untreated | (curcumin - 76.07%) | (curcumin - 99.2%) |
| Area after | 6048990 | 7092123 | 6231718 |
| scratching | | | |
| t = 0 | | | |
| Area after | 0 | 6079717 | 5295914 |
| t = 12 hrs | | | |
| Percentage | 100 | 14.2 | 15.01 |
| wound | | | |
| healing | | | |

Hemolytic toxicity in Dogs: This is a unique adverse reaction observed at concentrations equal to or greater the 20 mg/kg “first-in dog” with the liposomal curcumin. The physiopathologic cause appears to involve disruption of homeostatic mechanisms in the red blood cell membrane following exposure to liposome alone or the high concentration of pure curcumin or both. There are no reports of hemolysis with the three curcuminoids at 40 mg/kg (two times the canine hemolytic dose) in rats and mice (data not shown). Hence this adverse effect may be a heretofore unknown effect of pure curcumin or curcuminoids in specific to dog red blood cells. The observation of reversible hemolysis following exposure to liposomal curcumin at 20 mg/kg in dogs represents either an apparently distinct characteristic of the specific composition of the liposome, the use of pure curcumin at a toxic concentration or the combination of liposomes with curcumin. Preliminary toxicology data in a multi-dose liposomal curcumin trial in dogs revealed a clear dose related adverse event relationship not observed with curcumin at NOAEL dosages and not observed with clinically approved liposomal formulations of other drugs.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof' is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 7,220,438: Pharmacological Activities of Curcuma longa Extracts.

U.S. Patent Application Publication No. 20090047371: Pharmaceutical Composition Comprising Curcumin and Resveratrol and Uses Thereof in Medical Field.

U.S. Patent Application Publication No. 20100286585: Shunt Delivery of Curcumin.

1. Kim S J, Son T G, Park H R, Park M, Kim M S, Kim H S, Chung H Y, Mattson M P, Lee J (2008) Curcumin stimulates proliferation of embryonic neural progenitor cells and neurogenesis in the adult hippocampus. J Biol Chem 283(21): 14497-14505.

2. Anderson P, Morris R, Amaral 0, Bliss T and O'Keef Jed. (2007) The Hippocampus Book. Oxford University Press. ISBN 9780195100273.

3. Xu Y, Ku B, Cui L, Li X, Barish P A, Foster T C, and W O Ogle (2007) Curcumin reverses impaired hippocampal neurogenesis and increases serotonin receptor 1A mRNA and brain-derived neurotrophic factor expression in chronically stressed rats. Brain Research 1162:9-18.

4. Ray B, Bisht S, Maitra A, Maitra A, and Lahiri O K (2010) Neuroprotective and neurorescue effects of a novel polymeric nanoparticle formulation of curcumin (Nanocurc®) in the neuronal cell culture and animal model: implications for Alzheimer's Disease. J. Alzheimer's Disease, in press.

5. Kang S K, Cha S H, Jeon H G (2006) Curcumin-induced histone hypoacetylation enhances caspase-3 dependent glioma cell death and neurogenesis of neural progenitor cells. Stem Cells Dev. 15(2):165-174.

6. Lan Li et al (2005), Cancer 104 1322-1331.

7. Lan Li et al (2007) Mol Can Thera 6:1276-1282.

8. Mach C M et al (2009) J. Anticancer research 29:1895-1900.

9. Mach C M et al (2010) J. Anticancer Research 30:811-814.

10. Bisht S et al (2007) J. Nanobiotechnology 5:3.

11. Lim K J et al (2010) MCR AB #4440.

12. Bisht S et al (2010) Mol Can Ther.

13. Balmiki R al (2010) J. Alzheimers disease.

14. Vishwanatha J K 2009 Unither Conf, February.

15. Mukerjee A and Vishwanatha J K (2009) Anticancer Research 29:3867-3876.

16. Ranjan A P, Mukerjee A et al (2009) US-Jap Drug Delivery AB 1526.

17. Mukerjee A (2010) Curr Pharm Biotech 11:223-228.

18. Mukerjee A et al (2010) J Phys Chem 114: 12679-12684.

What is claimed is:

1. A composition for ameliorating symptoms or treating one or more proliferative disorders in a subject comprising:
one or more spherical liposomes comprising a lipid or a phospholipid wall, wherein the liposome encloses a synthetic curcumin (S-curcumin) or derivatives and modifications thereof dissolved or dispersed in an aqueous or a non-aqueous solvent with one or more optional related co-factors, proteins, antibodies, pain medications, and other pharmaceutically active agents dissolved, dispersed, or suspended in the solvent;
a suitable aqueous or non-aqueous dispersion medium, wherein the one or more spherical liposomes are dispersed in the dispersion medium; and
one or more optional excipients, diluents, extended or controlled release agents, lubricants, preservatives or any combinations thereof.

2. The composition of claim 1, wherein the one or more proliferative disorders are selected from at least one of a breast, uterine, cervical, ophthalmic, or pancreatic cancer.

3. The composition of claim 1, wherein the lipid or the phospholipid is selected from the group consisting of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl stearate, isopropyl myristate, amphoteric acrylic polymers, fatty acid, fatty acid amides, cholesterol, cholesterol ester, diacylglycerol, and diacylglycerolsuccinate.

4. The composition of claim 1, wherein the composition adapted for enteral, parenteral, intravenous, sub-cutaneous, intra-muscular, or intra-peritoneal injection in the subject.

5. The composition of claim 1, wherein the one or more liposomes have a size of about 100 nm.

6. A composition for ameliorating symptoms or treating one or more proliferative disorders in a subject comprising:
a biodegradable polymer conjugate dissolved or dispersed in a suitable aqueous or non-aqueous solvent, wherein the conjugate comprises a synthetic curcumin (S-curcumin) or derivatives and modifications thereof conjugated to one or more polymers selected from the group consisting of polyesters, polylactides, polyglycolides, polycaprolactones, polyanhydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), copolymers, terpolymers, and combinations or mixtures thereof; and
one or more excipients, diluents, extended or controlled release agents, lubricants, preservatives or any combinations thereof.

7. The composition of claim 6, wherein the one or more proliferative disorders are selected from at least one of a breast, uterine, cervical, ophthalmic, or pancreatic cancer.

8. The composition of claim 6, wherein the composition adapted for enteral, parenteral, intravenous, sub-cutaneous, intra-muscular, or intra-peritoneal injection in the subject.

9. The composition of claim 6, wherein the composition is used for the treatment of one or more neurological or neurodegenerative conditions selected from at least one of Parkinson's disease (PD), Alzheimer's disease, stress disorders, senile dementia, vascular dementias, Pick's disease, Creutzfeldt-Jacobs disease, and aging.

10. The composition of claim 6, wherein the synthetic curcumin is 85, 86, 87, 88, 89, 90, 91, 92, 93, 94, 95 or 96% pure diferuloylmethane.

11. A method of ameliorating symptoms or treating one or more proliferative disorders in a subject comprising the steps of:
identifying the subject in need of amelioration of symptoms or treatment of the one or more proliferative disorders; and
administering intravenously one or more pharmaceutical compositions comprising a therapeutically effective amount of a synthetic curcumin (S-curcumin) or derivatives and modifications thereof dissolved or dispersed in a suitable aqueous or non-aqueous medium, wherein the curcumin is enclosed in one or more spherical liposomes or is conjugated to one or more biodegradable polymers.

12. The method of claim 11, wherein the one or more proliferative disorders are selected from at least one of a breast, uterine, cervical, ophthalmic, or pancreatic cancer.

13. The method of claim 11, wherein the liposomes comprise a lipid or a phospholipid wall.

14. The method of claim 13, wherein the lipid or the phospholipid is selected from the group consisting of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl stearate, isopropyl myristate, amphoteric acrylic polymers, fatty acid, fatty acid amides, cholesterol, cholesterol ester, diacylglycerol, and diacylglycerolsuccinate.

15. The method of claim 11, wherein the therapeutically effective amount comprises 50 nM/kg, 10 to 100 nM/kg, 25 to 75 nM/kg, 10, 20, 30, 40, 50, 60, 70, 80, 90, or 100 nM/kg of body weight of the subject.

16. The method of claim 11, wherein the synthetic curcumin is 85, 86, 87, 88, 89, 90, 91, 92, 93, 94, 95 or 96% pure diferuloylmethane.

17. A composition for ameliorating symptoms or treating breast cancer in a human subject comprising synthetic curcumin (S-curcumin) or derivatives and modifications thereof dissolved or dispersed in a suitable aqueous or non-aqueous medium, wherein the S-curcumin is enclosed in one or more spherical liposomes or is conjugated to one or more biodegradable polymers.

18. The composition of claim 17, wherein the liposome comprises a lipid or phospholipid, wherein lipid or the phospholipid is selected from the group consisting of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl stearate, isopropyl myristate, amphoteric acrylic polymers, fatty acid, fatty acid amides, cholesterol, cholesterol ester, diacylglycerol, and diacylglycerolsuccinate.

19. The composition of claim 17, wherein the biodegradable polymer is selected from the group consisting of polyesters, polylactides, polyglycolides, polycaprolactones, polyanhydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), copolymers, terpolymers, and combinations or mixtures thereof

20. The composition of claim 17, wherein the composition adapted for intravenous, sub-cutaneous, intra-muscular, or intra-peritoneal injection in the subject.

21. The composition of claim 17, wherein the synthetic curcumin is 85, 86, 87, 88, 89, 90, 91, 92, 93, 94, 95 or 96% pure diferuloylmethane.

22. A composition for ameliorating symptoms or treating at least one of proliferative disorders, neurological disorders, or neurodegenerative conditions, in a subject comprising: curcumin, synthetic curcumin (S-curcumin), curcumin analogues, curcumin derivatives or combinations thereof dissolved or dispersed in a suitable aqueous or non-aqueous medium, wherein the curcumin or the S-curcumin is enclosed in one or more spherical liposomes or is conjugated to one or more biodegradable polymer, wherein the liposome comprises a lipid or phospholipid, wherein lipid or the phospholipid is selected from the group consisting of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl stearate, isopropyl myristate, amphoteric acrylic polymers, fatty acid, fatty acid amides, cholesterol, cholesterol ester, diacylglycerol, and diacylglycerolsuccinate and wherein the biodegradable polymer is selected from the group consisting of polyesters, polylactides, polyglycolides, polycaprolactones, polyanhydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), copolymers, terpolymers, and combinations or mixtures thereof.

23. The composition of claim 22, wherein the one or more proliferative disorders are selected from at least one of a breast, uterine, cervical, ophthalmic, or pancreatic cancer.

24. The composition of claim 22, wherein the neurological disorder or the neurodegenerative condition is selected from at least one of Parkinson's disease (PD), Alzheimer's disease, stress disorders, senile dementia, vascular dementias, Pick's disease, Creutzfeldt-Jacobs disease, and aging.

25. The composition of claim 22, wherein the composition adapted for enteral, parenteral, intravenous, sub-cutaneous, intra-muscular, or intra-peritoneal injection in the subject.

26. The composition of claim 22, wherein the composition is administered along with related co-factors, proteins, antibodies, pain medications, and other pharmaceutically active agents selected from at least one of L-dopa, Carbidopa, benserazide, Tolcapone, dopamine agonists bromocriptine, pergolide, pramipexole, ropinirole, piribedil, cabergoline, apomorphine, lisuride, MAO inhibitors, serotonin reuptake inhibitors, sertraline, paroxetine, selegiline, or rasagiline.

27. The composition of claim 22, wherein the synthetic curcumin is 85, 86, 87, 88, 89, 90, 91, 92, 93, 94, 95 or 96% pure diferuloylmethane.

28. A method of ameliorating symptoms or treating one or more proliferative disorders, neurological disorders, neurodegenerative conditions, or any combinations thereof in a subject comprising the steps of:
identifying the subject in need of amelioration of symptoms or treatment of the one or more proliferative disorders, neurological disorders, neurodegenerative conditions, or any combinations thereof; and
administering intravenously one or more pharmaceutical compositions comprising a therapeutically effective amount of curcumin, synthetic curcumin (S-curcumin), curcumin analogues, curcumin derivatives or combinations thereof dissolved or dispersed in a suitable aqueous or non-aqueous medium, wherein the curcumin or the S-curcumin is enclosed in one or more spherical liposomes or is conjugated to one or more biodegradable polymers.

29. The method of claim 28, wherein the neurological disorder or the neurodegenerative condition is selected from at least one of Parkinson's disease (PD), Alzheimer's disease, stress disorders, senile dementia, vascular dementias, Pick's disease, Creutzfeldt-Jacobs disease, and aging.

30. The method of claim 28, wherein the synthetic curcumin is 85, 86, 87, 88, 89, 90, 91, 92, 93, 94, 95 or 96% pure diferuloylmethane.
